(19) United States
(12) Patent Application Publication
(10) Pub. No.: US 2007/0135845 A1
(43) Pub. Date: Jun. 14, 2007

(54) Method and devices for utilizing physiologic and biologic electrical potentials

(76) Inventors: Hans A. Mische, St. Cloud, MN (US); Eugene F. Mische, St. Cloud, MN (US)

Correspondence Address: HANS MISCHE, 32 HIGHBANKS PLACE, ST. CLOUD, MN, 56301, US

(21) Appl. No.: 11/602,714

(22) Filed: Nov. 21, 2006

Related U.S. Application Data
(60) Provisional application No. 60/738,892, filed on Nov. 22, 2005.

(51) Int. Cl. A61N 1/00

(52) U.S. Cl. 607/2

(57) ABSTRACT

Methods and devices that utilize various physiologic and biologic electrical potentials and material properties in order to power complementary devices in order to provide therapeutic and or diagnostic capabilities. Alternatively, the electrical energy can be directly applied to the target tissues for the therapeutic or diagnostic intent.

### CROSS REFERENCE TO RELATED APPLICATION

This application claims the benefit of U.S. Provisional applications having Ser. No. 60/738,892, filed Nov. 22, 2005, and Ser. No. 60/171,687 filed on Dec. 21, 1999, U.S. Pat. No. 6,375,666, U.S. Pat. No. 6,764,498, U.S. Provisional Ser. No. 11/504,514, and U.S. Provisional Ser. No. 10/843,828 which are all incorporated herein by reference in their entirety.

### FIELD OF INVENTION

The present invention relates generally to the utilization of the human physiology as a platform and means of generating electromagnetic energy. The energy generated is subsequently utilized to power therapeutic or diagnostic devices, or to deliver therapeutic treatment or diagnostic methods directly. Methods and devices are disclosed in the context of generating electromagnetic energies for the treatment or diagnosis of physiologic ailments and conditions.

### BACKGROUND OF INVENTION

Currently, there are many implantable therapeutic and diagnostic device that require a source of operational energy in the form of a battery. Many times the battery needs to be replaced or recharged. This can require surgery to access the device in order to replace the battery or at least a visit to a medical facility in order to recharge the battery. Disclosed are methods and devices that can render obsolete the need for batteries as currently perceived, or to provide means of treatment and/or diagnosis.

### SUMMARY

In contrast to the current prior art, the present invention proposes utilizing using the inherent or resident physiologic and biologic properties of the anatomy to provide power a variety of therapeutic or diagnostic devices in vivo. The current prior art requires an external or integral battery as a source of power. These batteries have a predetermined life-span and need replacement or recharging. The present invention will render obsolete the need for batteries for many applications or delay the need to replace or recharge the batteries. In addition, the present invention will provide means of creating closed loop circuitry within biologic tissues and fluids. These devices can be implanted within the body or attached to the external surface of the body. The devices can be constantly operational or only periodically activated by certain physiologic or biologic activity or requirement.

The invention is disclosed in the context of operating medical devices for various clinical indications, but applications are contemplated as well. For example, a radio, telephone, transmitter, or a receiver can be powered by physiologic or biologic parameters and activities. Visible light sources may be in whole or partially activated by various physiologic and or biologic parameters and activities. Other applications that can benefit from the present invention are anticipated.

### BRIEF DESCRIPTION OF THE DRAWINGS

Throughout the several views of the drawings several illustrative embodiments of the invention are disclosed. It should be understood that various modifications of the embodiments might be made without departing from the scope of the invention. Throughout the views identical reference numerals depict equivalent structure wherein:

FIG. 1 is a schematic diagram of an inductive device activated by flowing fluids

FIG. 2 is a schematic diagram of a thermocouple powered generator implanted within the brain

FIG. 3 is a schematic diagram of the head with a pH probe powered circuit implanted within the thalamic region of the brain

FIG. 4 is a schematic diagram of a galvanic cell within biologic electrolyte

FIG. 5 is a schematic diagram of a cylindrical galvanic cell arrangement

### DETAILED DESCRIPTION

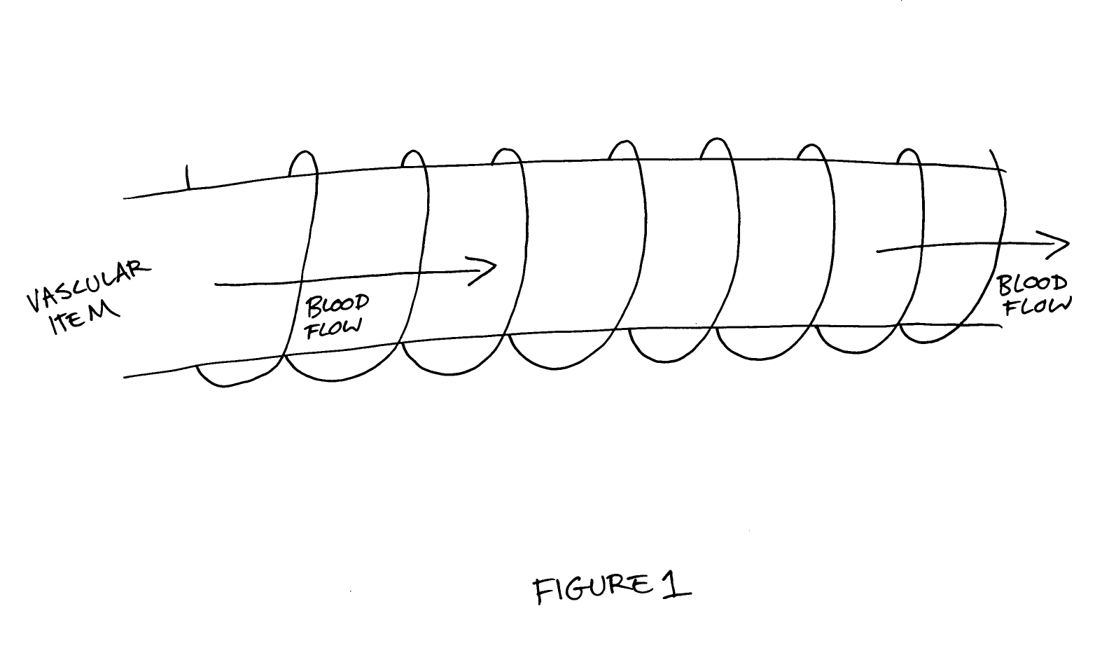

Referring now to the figures, FIG. 1 shows a device that is activated by fluids which flow through the tubular structure. The device can be fashioned similarly to an intravascular stent that are now commonly in use. The stent, if designed similarly like a classic inductor in a coil fashion, it can serve to purposes. It can keep an artery open as well as operate as a component in an inductive circuit. When blood flows, or other fluids, flow through the structure, voltages, currents and magnetic fields are produced. If electrical connections with leads are made to the structure, these electrical parameters can then be harnessed to power devices or they can be applied directly to anatomical tissues for treatment or diagnostic purposes.

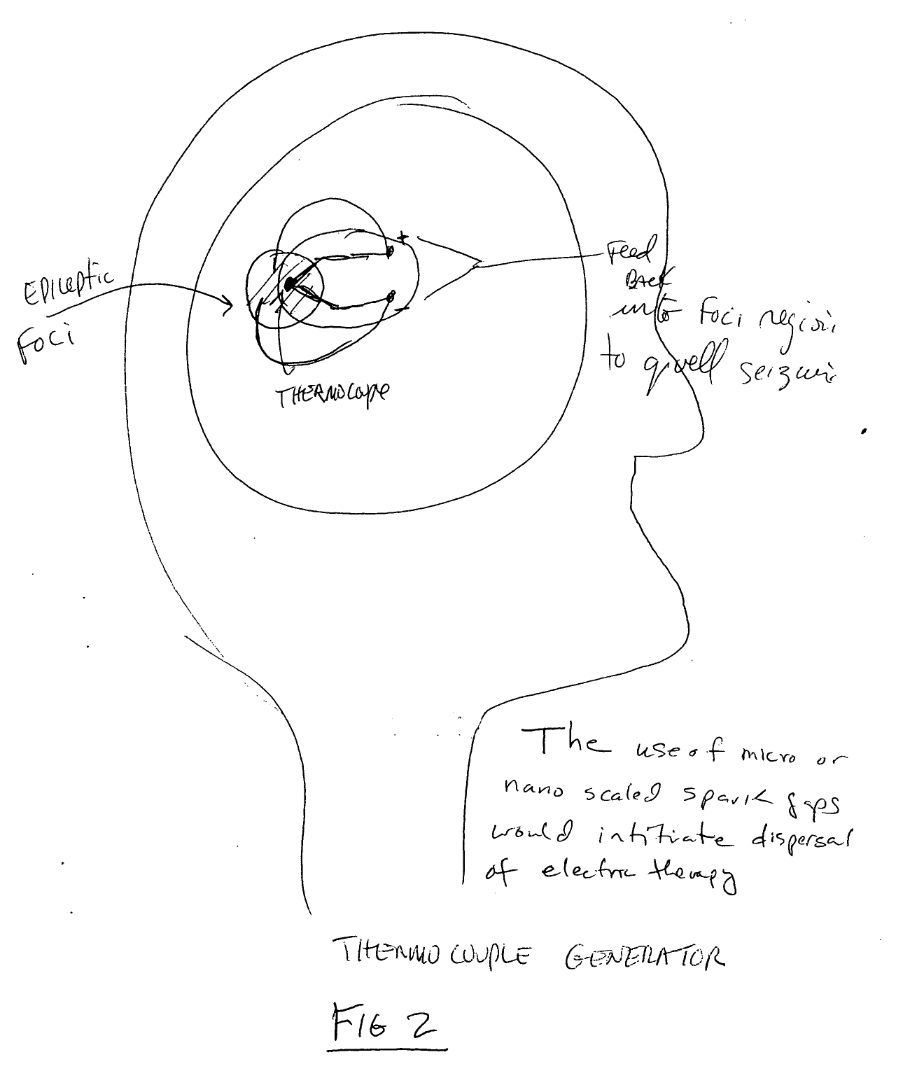

FIG. 2 shows a thermocouple device that is implanted within the brain at a location where epileptic seizures occur. In this case, the patient may be suffering from epilepsy. When the seizures occur at the epileptic foci, the localized temperatures increase due to the increase in brain activity. The change of temperatures generates a voltage and resultant current flow within the thermocouple probe which can be used to power a device circuit that would discharge into the seizure foci and stop the seizure. In another embodiment, the thermocouple delivers the electrical parameters directly back into the foci immediately. Multiple thermocouples can be joined in order to create a thermopile. In exploiting the temperature changes of anatomical tissues, pyroelectric materials and circuitry can also be utilized to generate voltages and currents. Devices can be placed in various tissues or organs. If placed within the esophagus, the respiration cycle would alternately cool and warm the devices and cause a cyclic generation of electrical energy. If applied to the surface of the body, both environmental and anatomic temperature changes would cause the pyroelectric devices to activate.

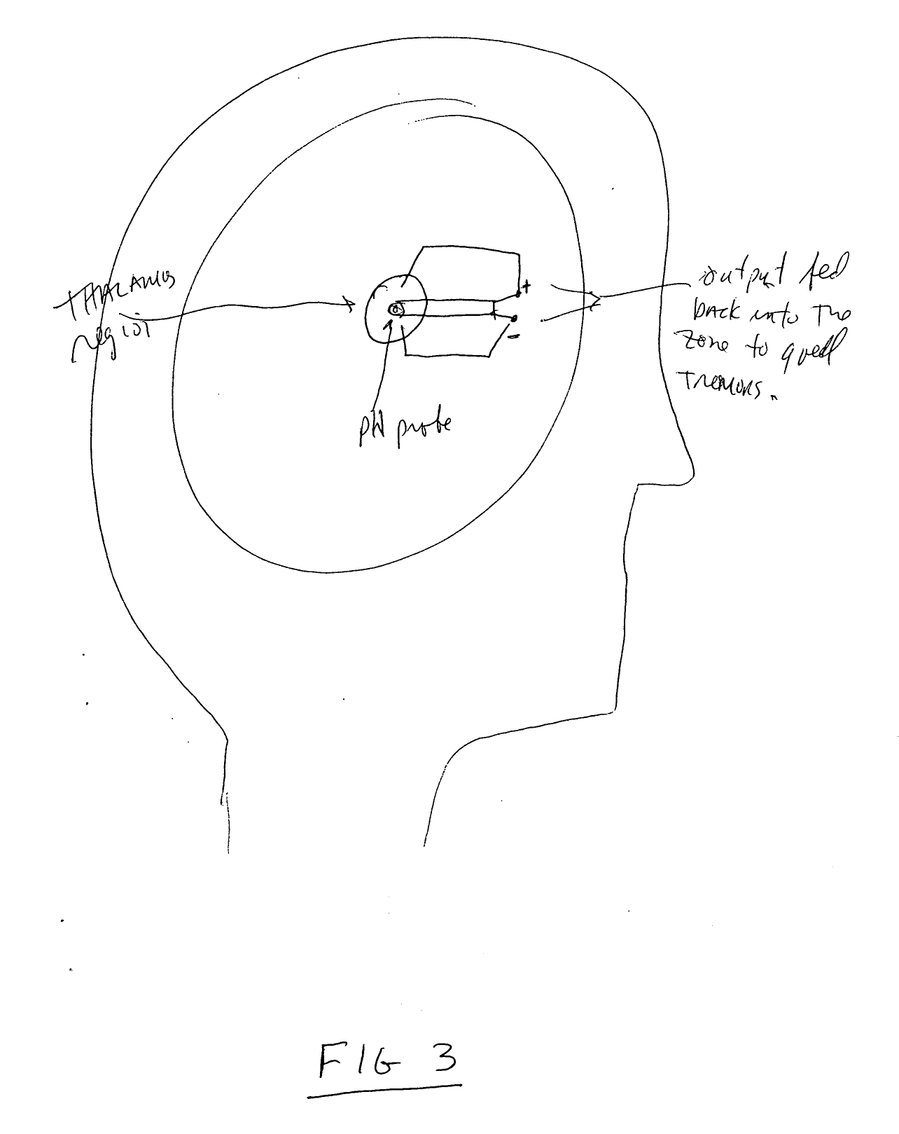

FIG. 3 shows a pH probe circuit implanted within the thalamic region of the brain for the treatment of Parkinson's or Essential Tremor. In the case of Parkinson's, the thalamic region becomes electrically and electrochemically overactive, causing tremors. This over activity generates enzymes and chemical bi-products that affect the localized pH of the tissues. As the pH changes, the pH probe becomes activated and the current is activated by the resultant voltage and current production. The circuit can thus charge to a level required to deactivate the thalamic region. When this level is reached, the circuit discharges directly into the thalamic region and deactivates the active tissues and arrests the seizures. A circuit like this or any of the aforementioned and subsequent circuits and devices can be used to charged complementary generators, stimulators, pumps, and other therapeutic and diagnostic constructs.

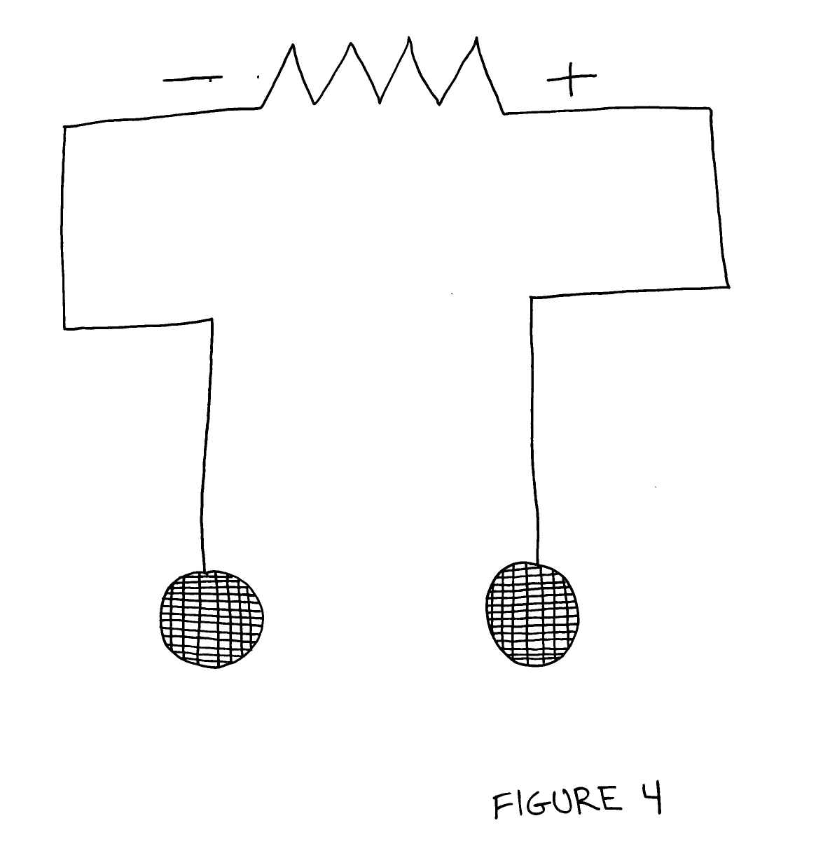

FIG. 4 shows a galvanic cell surrounded by the natural electrolytic fluids within the anatomy. These fluids 43 provide a necessary element for the construction of this galvanic cell. The cell also consists of an anode 41 and a cathode 42. The load 44, can be another circuit that is powered by the cell, or the load may actually be a direct connection from the cell to the anatomy. In this case, a constant voltage and current can be consistently applied to a specific site to act as a preventative or prophylactic treatment. In the case of epilepsy or depression, it would mean that the formation of a clinical event (seizure or depression) would possibly never happened or kept to a minimum level.

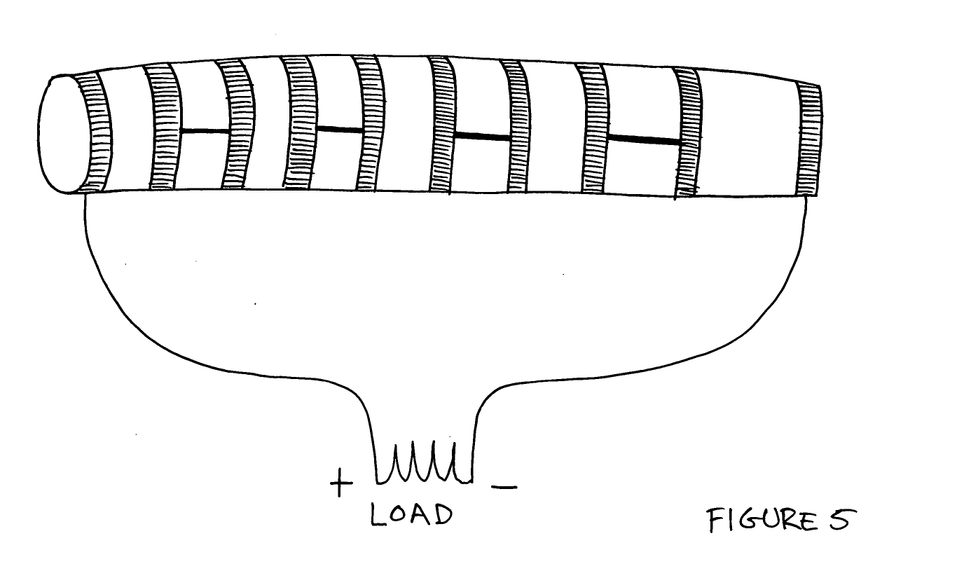

FIG. 5 is an alternative embodiment of a galvanic cell construction. In this case, it is a cylindrical structure with an array of electrodes that are ganged together. The structure can also be made in a tubular or stent like arrangement so that it can hold open vessels, ventricles, conduits, intestinal tracts, or other anatomical structures. This is another situation similar to that in FIG. 1 where the device serves multiple purposes.

Another embodiment, as disclosed in U.S. Provisional Application Ser. No. 60/171,687 file on Dec. 21, 1999, piezoelectric materials can be used to leverage the mechanical motion of the anatomy to generate electrical energy. For example, if a piezoelectric element is attached to the beating heart, the bending of the element will generate electrical energy that can then be used directly for therapies or to activate or charge other devices. Additionally, the piezoelements can be attached or placed within other organs or anatomy such as, but not limited to, the lungs, diaphragm, esophagus, stomach, intestines, muscles, joints, eyes, and bladder. Accelerometers can also be used in a similar fashion.

The tissues of the body have natural electrical resistive, impedance, inductive and capacitive properties. These properties all can be exploited as integral components of circuitry. For example, a device can be built that would isolate a volume of tissue which could then be a capacitor that is capable of holding an electrical charge. This charge can then be discharge when needed. The capacitive tissue can also be utilized in an LCR timing circuit. Likewise, a certain volume of tissue can represent an impedance or resistive load within an circuit embedded or attached to tissues. Vessels and bones can become inductors or complex impedances.

What is claimed:

1. A method of generating operational energy by:
utilizing the electrolytes within physiologic tissues to complete a battery circuit

2. A method of generating operational energy by:
utilizing the blood flow though a inductive device to generate therapeutic electric and magnetic fields

3. A method of generating operational energy by:
attaching a piezoelectric element to a movable anatomical structure, resultant movement causing deflection of piezoelectric element, deflection generating electrical energy, electrical energy being used for therapeutic event fields
